(19) United States
(12) Patent Application Publication
(10) Pub. No.: US 2026/0193227 A1
(43) Pub. Date: Jul. 9, 2026

(54) THERAPEUTIC COMPOUNDS AND METHODS

(71) Applicant: UNIVERSITY OF IOWA RESEARCH FOUNDATION, Iowa City, IA (US)

(72) Inventors: Michael Ashley Spies, Iowa City, IA (US); Maria Spies, Iowa City, IA (US)

(73) Assignee: UNIVERSITY OF IOWA RESEARCH FOUNDATION, Iowa City, IA (US)

(21) Appl. No.: 19/131,890

(22) PCT Filed: Nov. 21, 2023
(86) PCT No.: PCT/US2023/080790

§ 371 Date: May 21, 2025

Related U.S. Application Data
(60) Provisional application No. 63/427,759, filed on Nov. 23, 2022.

(51) Int. Cl. C07D 401/06; A61K 31/18; A61K 31/4439; A61P 35/00; C07C 311/21

(52) U.S. Cl. CPC C07D 401/06; A61K 31/18; A61K 31/4439; A61P 35/00; C07C 311/21

(57) ABSTRACT

The invention provides a compound of formula (I) or (II): or a salt thereof, wherein X, Y, R¹, R², and R³ have any of the values described in the specification, as well as compositions comprising a compound of formula I. The compounds are useful as RAD52 inhibitors and as anticancer agents.

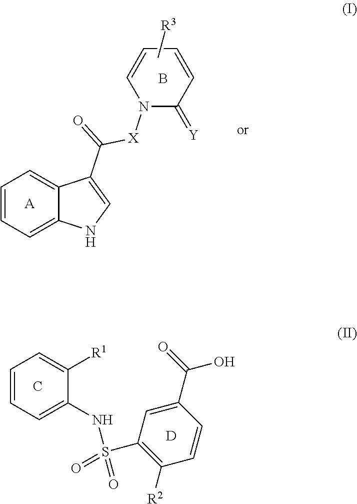

### CROSS-REFERENCE TO RELATED APPLICATION

This application claims priority to U.S. Provisional Application No. 63/427,759 that was filed on Nov. 23, 2022. The entire content of the application referenced above is hereby incorporated by reference herein.

### GOVERNMENT FUNDING

This invention was made with government support under CA232425 awarded by the National Institutes of Health. The government has certain rights in this invention.

### BACKGROUND

Human DNA repair protein RAD52 (Mortensen U H, et al., Current Biology. 2009; 19(16):R676-R7; and Spies M. Curr Opin StructBiol. 2013; 23(1):154-60), is a validated target for the development of pharmacological inhibitors that can be used as single agents or in combination with other therapies in cancers displaying BRCAness phenotype, as well as ATM (ataxia-telangiectasia mutated) deficient cancers (Bhat D S, et al., DNA Repair. 2022; 120:103421; Cramer-Morales K, Blood. 2013; 122(7):1293-304; Lok B H, et al., Oncogene. 2013; 32(30):3552-8, Feng Z, et al., Proc Natl Acad Sci USA. 2011; 108(2):686-91; Rossi M J, Frontiers in Genetics. 2021; 1; Hengel S R, eLife. 2016; 5; Chheda P, Spies M, Spies M A. Burger's Medicinal Chemistry andDrug Discovery. p. 1-39; and Treuner K, et al., Oncogene. 2004; 23(27):4655-61). Previous work showed that pharmacological inhibition of RAD52 is selectively toxic to cells carrying biallelic defects in BRCA1, BRCA2, and PALB2 (Hengel S R, eLife. 2016; 5; Chandramouly G, et al., PloS one. 2016; 11(1); Sullivan K, et al., PloS one. 2016; 11(1); and Huang F, et al., Nucleic Acids Res. 2016; 44(9):4189-99), and cell deficient in ATM serine/threonine kinase (Treuner K, et al., Oncogene. 2004; 23(27):4655-61). On the other hand, RAD52 inhibition in normal, BRCA- and ATM-proficient cells is not expected to cause toxicity, as RAD52 inactivation due to mutations is well tolerated ((Rijkers T, et al., Mol Cell Biol. 1998; 18(11):6423-9; Yamaguchi-Iwai Y, et al., Mol Cell Biol. 1998; 18(11):6430-5; and Yanez R J, Porter A C. DNucleic Acids Res. 2002; 30(3):740-8) and LOEUF score of 0.64 at gnomAD database). Situation is unique among other DNA repair and genome maintenance proteins that share synthetic lethality with BRCA deficiency (see (3) for review). RAD52 does not possess any bonafide enzymatic activities, but is involved in numerous protein-nucleic acid (PNI) and protein-protein interactions (PPI). Since many of RAD52's cellular functions depend on its capacity to bind ssDNA, ssRNA, single-stranded features of complex DNA substrates, or ssDNA bound by Replication Protein A, RPA, the main ssDNA binding protein in eukaryotic cells (Bhat D S, et al., DNA Repair. 2022; 120:103421; Malacaria E, et al., Cancers. 2020; 12(2); and Caldwell C C, Spies M. Critical reviews in biochemistry and molecular biology. 2020; 55(5):482-507), inhibiting the RAD52-ssDNA complexation represents a validated target for developing small molecule inhibitors that would interfere with the cellular functions of RAD52. Efficient pharmacological targeting of the protein-DNA interactions with small molecules presents a formidable challenge due to the nature of the DNA binding sites on proteins. RAD52 binds ssDNA and displays no sequence specificity. The narrow continuous binding cavity of the RAD52 ssDNA binding site spans the circumference of the protein oligomeric ring (Grimme J M, et al., Nucleic Acids Res. 2010; 38(9):2917-30; Honda M, et al., The EMBO journal. 2011; 30(16):3368-82; Kagawa W, et al., Mol Cell. 2002; 10(2):359-71; Saotome M, et al., iScience. 2018; 3:50-62; and Singleton M R, et al., Proc Natl Acad Sci USA. 2002; 99(21):13492-7). An effective and specific inhibitor of the RAD52-ssDNA interaction, therefore needs to outcompete a strong multivalent RAD52-ssDNA interaction, while at the same time displaying specificity for RAD52 over multitude of cellular ssDNA binding proteins, such as RPA. RAD52 ssDNA binding grove offers a repetitive pattern of potential small molecule binding pockets reasonable druggability scores (Hengel S R, eLife. 2016; 5).

Hengel, S. R., et al., 2016; eLife, 5 found a variety of small molecule natural products in a small HTS screen of the Microsource Spectrum library. In a parallel in silico, structure-based analysis a predictive pharmacophoric model of RAD52 inhibition was constructed. This structural model was highly successful in identifying novel natural products that bind to RAD52 and inhibit its interaction with ssDNA. An important aspect of that study was that epigallochatechin (EGC) had a very desirable selectivity, in that it competed with ssDNA binding to RAD52, but not to RPA. It was also effective in BRCA2-depleted cells confirming that pharmacological inhibition of RAD52-ssDNA interaction can perform the same function as RAD52 depletion. Selectivity for RAD52 over RPA was a unique property of EGC among potent compounds identified in the HTS screen. The structural models indicated that EGC binds to a distinct hot spot within the RAD52 DNA binding groove, suggesting that future scaffold hopping into drug like space should primarily focus on this region.

Currently there is a need for agents that are useful for inhibiting RAD52. Such agents would be useful for treating cancer, alone, or in combination with other cancer therapies.

### SUMMARY

In one aspect the present invention provides compounds that are useful for inhibiting RAD52. The compounds are useful for treating cancer, alone, or in combination with other cancer therapies.

Accordingly, in one aspect, the invention provides a compound of formula (I) or (II):

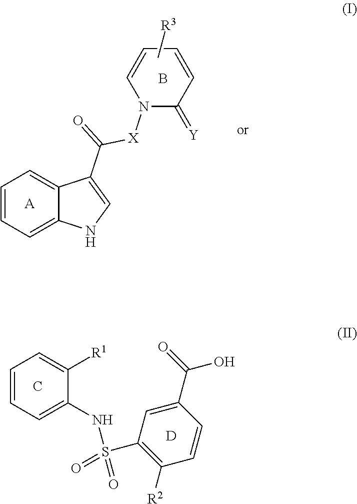

or a salt thereof, wherein:
   X is (C₁-C₃)alkenyl that is optionally substituted with one or more fluoro;
   Y is ═S or ═O;
   R¹ is H, (C₁-C₆)alkyl, or (C₁-C₆)alkoxy, wherein any (C₁-C₆)alkyl, and (C₁-C₆)alkoxy is optionally substituted with one or more groups independently selected from the group consisting of halo;
   R² is H, (C₁-C₆)alkyl, or (C₁-C₆)alkoxy, wherein any (C₁-C₆)alkyl, and (C₁-C₆)alkoxy is optionally substituted with one or more groups independently selected from the group consisting of halo;
   R³ is nitro, halo, hydroxy, cyano, carboxy, (C₁-C₆)alkyl, (C₁-C₆)alkoxy, (C₁-C₆)alkoxycarbonyl, —NR^{a}R^{b}, —S(═O)₂R^{c}, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups;
   ring A and ring B in formula (I) are each optionally substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy;
   ring C and ring D in formula (II) are each optionally substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy;
   R^{a} is H, (C₁-C₆)alkyl, (C₁-C₆)alkoxy, (C₁-C₆)alkoxycarbonyl, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups;
   R^{b} is H, (C₁-C₆)alkyl, (C₁-C₆)alkoxy, (C₁-C₆)alkoxycarbonyl, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups; and
   R^{c} is (C₁-C₆)alkyl that is optionally substituted with one or more fluoro groups.

The invention also provides a pharmaceutical composition comprising a compound of formula (I) or formula (II) or a pharmaceutically acceptable salt thereof, and a pharmaceutically acceptable excipient.

The invention also provides a method for treating or preventing cancer in an animal (e.g., a mammal such as a human) comprising administering compound of formula (I) or formula (II) or a pharmaceutically acceptable salt thereof to the animal.

The invention also provides a compound of formula (I) or formula (II) or a pharmaceutically acceptable salt thereof for use in medical therapy.

The invention also provides a compound of formula (I) or formula (II) or a pharmaceutically acceptable salt thereof for the prophylactic or therapeutic treatment of cancer.

The invention also provides the use of a compound of formula (I) or formula (II) or a pharmaceutically acceptable salt thereof to prepare a medicament for treating cancer in an animal (e.g. a mammal such as a human).

The invention also provides processes and intermediates disclosed herein that are useful for preparing a compound of formula I or a salt thereof.

### BRIEF DESCRIPTION OF THE FIGURES

FIG. 1. Identification of RAD52 with new chemical scaffolds. A. Workflow for scaffold hopping from the EGC-RAD52 complex. Three million compound ENAMINE REAL database was narrowed to 7,700 based on shape similarity to EGC and docked into the EGC pocket within the RAD52 ssDNA binding site. Two methods were used to score the compounds: In the first approach (orange flow) CCG/MOE PLIF tool was used to create fingerprints for RAD52-ligand interactions, in order to find compounds mimicking the contacts of EGC and RAD52; in the second approach (green flow) the London dG scoring function was used to rank order compounds. Three top scoring compounds from each method were ordered, with compounds Z991573902 (Z99, a PLIF hit, compound 1), and Z56785913 (Z56, a docking hit, compound 8) displaying promising activities in vitro and in cells B. Ligand maps for EGC and compounds Z99, and Z56. The interaction key is shown below. Among notable contacts, all three compounds interact with R55, one of the key residues in the DNA binding site of RAD52. In contract to EGC, however, whose interactions are dominated by van der Waals interaction and water-mediated contacts, Z99 and Z56 are involved in more direct interactions including hydrogen bonding.

FIG. 2. New synthetic compound, Z991573902 (Z99, compound 1) binds in the ssDNA binding grove of the RAD52 oligomeric ring, inhibits the RAD52-ssDNA interaction and shows toxicity towards BRCA2-mutated cells. A. Ligand map for the Z99-RAD52 complex. A key ssDNA binding residue R55 is marked with an asterisk. The interaction key is shown in FIG. 1B. B. Ligand map for the Z99-RPA complexation. Z99 can form a complex with the DNA binding domain A of RPA1. Three ssDNA binding residues of RPA are marked by asterisks. C.-E. The in vitro FRET-based assays follow inhibition of the RAD52-ssDNA interaction (C.), RPA-ssDNA interactions (D.), and the interaction between RAD52 and RPA-ssDNA complex (E.) by Z99. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of varying concentrations of Z99 was evaluated using CellTiter-Glo assay. The data are plotted as average±standard deviation for at least three independent measurements. Hundred percent live cells corresponds to DMSO only control. Note that above 100 μM, Z99 becomes insoluble in cell culture medium. H. Representative wells from the clonogenic survival assay at different concentrations of Z99.

FIG. 3. New synthetic compound, Z56785913 (Z56, compound 8) binds in the ssDNA binding grove of the RAD52 oligomeric ring, shows selectivity towards RAD52 over RPA, and is selectively toxic to BRCA2-mutated cells. A. Ligand map for the Z56-RAD52 complex. A key ssDNA binding residue R55 is marked with an asterisk. The interaction key is shown in FIG. 1B. B. There was no interaction between Z56 and RPA. C.-E. The in vitro FRET-based assays follow inhibition of the RAD52-ssDNA interaction (C.), RPA-ssDNA interactions (D.), and the interaction between RAD52 and RPA-ssDNA complex (E.) by Z56. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z56 was evaluated using CellTiter-Glo assay. The data are plotted as average±standard deviation for at least three independent measurements. Hundred percent live cells corresponds to DMSO only control. Note that above 100 μM, Z99 becomes insoluble in cell culture medium. H. Representative wells from the clonogenic survival assay at different concentrations of Z56.

FIG. 4. Six new compounds were designed based on the Z99 scaffold. In order to enhance the affinity and specificity of our inhibitors, we selected Z99 as the query in a large search of chemical space using the program InfiniSee (BiosolveIT). This program works not by searching chemical libraries for similarity to the query, but rather by a combinatorial fragment build-up scheme, based on predetermined number of chemical reactions and defined fragments (the rules which circumscribe the chemical space). The REAL (Enamine) chemical space is vast, consisting of 31,612,189,569 (~3.2×1010) virtual products; based on 165 Enamine reactions, 100,526 in-stock reagents, and 182,381 selected building blocks. Six compounds (compounds 2-7) were selected based on the diversity of the generated scaffolds and were synthesized to >99% purity by Enamine. All six compounds have improved capacity to inhibit RAD52, while displaying improved selectivity towards RAD52 over RPA.

FIG. 5. New synthetic compound, a derivative of the Z99 scaffold Z5862216254 (Z58-54, 2) binds in the ssDNA binding grove of the RAD52 oligomeric ring, shows selectivity towards RAD52 over RPA, and is selectively toxic to BRCA2-mutated cells. A. Ligand map for the Z58-54/RAD52 complex. Two key ssDNA binding residues, R55 and K152 are marked with asterisks. The interaction key is shown in FIG. 1B. B. Ligand map for the Z58-54/RPA complexation. The key ssDNA binding residue F238 is marked with asterisk. Note the reduced number of contacts compared to the parental scaffold. C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-54. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-54 was evaluated using CellTiter-Glo assay. The data are plotted as average±standard deviation for at least three independent measurements. Hundred percent live cells corresponds to DMSO only control.

FIG. 6. New synthetic compound, a derivative of the Z99 scaffold Z5862231762 (Z58-62, 3) binds in the ssDNA binding grove of the RAD52 oligomeric ring, shows selectivity towards RAD52 over RPA, and is selectively toxic to BRCA2-mutated cells. A. Ligand map for the Z58-62/RAD52 complex. Two key ssDNA binding residues, R55 and K152 are marked with asterisks. The interaction key is shown in FIG. 1B. B. Ligand map for the Z58-62/RPA complexation. The key ssDNA binding residues F238 and F269 are marked with asterisks. Note the reduced number of contacts compared to the parental scaffold. C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-62. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-62 was evaluated using CellTiter-Glo assay. The data are plotted as average±standard deviation for at least three independent measurements. Hundred percent live cells corresponds to DMSO only control.

FIG. 7. New synthetic compound, a derivative of the Z99 scaffold Z5862148469 (Z58-69, 4) binds in the ssDNA binding grove of the RAD52 oligomeric ring, and shows selectivity towards RAD52 over RPA. A. Ligand map for the Z58-69/RAD52 complex. Two key ssDNA binding residues, R55 and K152 are marked with asterisks. The interaction key is shown in FIG. 1B. B. Ligand map for the Z58-69/RPA complexation. The key ssDNA binding residue located in the DBD-B of RPA, W361 is marked with an asterisk. Note the reduced number of contacts compared to the parental scaffold and different position of the compound. C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-69. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-69 was evaluated using CellTiter-Glo assay. The data are plotted as average±standard deviation for at least three independent measurements. Hundred percent live cells corresponds to DMSO only control. Note that this compound was poorly soluble in cell culture medium and failed to inhibit both BRCA2-mutated and BRCA2-complemented cells.

FIG. 8. New synthetic compound, a derivative of the Z99 scaffold Z58662209772 (Z58-72, 5) binds in the ssDNA binding grove of the RAD52 oligomeric ring, shows selectivity towards RAD52 over RPA, and is selectively toxic to BRCA2-mutated cells. A. Ligand map for the Z58-72/RAD52 complex. Two key ssDNA binding residues, R55 and K152 are marked with asterisks. The interaction key is shown in FIG. 1B. B. Ligand map for the Z58-72/RPA complexation. The key ssDNA binding residue located in the DBD-B of RPA, W361 is marked with an asterisk. Note the reduced number of contacts compared to the parental scaffold and different position of the compound. C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-72. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-72 was evaluated using CellTiter-Glo assay. The data are plotted as average±standard deviation for at least three independent measurements. Hundred percent live cells corresponds to DMSO only control. Note that this compound was poorly soluble in cell culture medium at concentrations higher than 10 μM. H. Representative wells from the clonogenic survival assay at different concentrations of Z58-72.

FIG. 9. New synthetic compound, a derivative of the Z99 scaffold Z58662135983 (Z58-83, 6) binds in the ssDNA binding grove of the RAD52 oligomeric ring, shows selectivity towards RAD52 over RPA, and is selectively toxic to BRCA2-mutated cells. A. Ligand map for the Z58-83/RAD52 complex. Two key ssDNA binding residues, R55 and K152 are marked with asterisks. The interaction key is shown in FIG. 1B. B. Ligand map for the Z58-83/RPA complexation. The key ssDNA binding residue located in the DBD-A of RPA, F2391 is marked with an asterisk. Note the reduced number of contacts compared to the parental scaffold C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-83. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-83 was evaluated using CellTiter-Glo assay. The data are plotted as average±standard deviation for at least three independent measurements. Hundred percent live cells corresponds to DMSO only control. Note that this compound was poorly soluble in cell culture medium at concentrations higher than 10 μM. H. Representative wells from the clonogenic survival assay at different concentrations of Z58-72.

FIG. 10. New synthetic compound, a derivative of the Z99 scaffold Z58662219887 (Z58-87, 7) binds in the ssDNA binding grove of the RAD52 oligomeric ring, shows selectivity towards RAD52 over RPA, and is selectively toxic to BRCA2-mutated cells. A. Ligand map for the Z58-87/RAD52 complex. Two key ssDNA binding residues, R55 and K152 are marked with asterisks. The interaction key is shown in FIG. 1B. B. Ligand map for the Z58-87/RPA complexation. The key ssDNA binding residues located in the DBD-A of RPA, F238 and F269 are marked with asterisks. Note the reduced number of contacts compared to the parental scaffold and different position of the compound. C.-E. The in vitro FRET-based assays follow inhibition of the RAD52-ssDNA interaction (C.), RPA-ssDNA interactions (D.), and the interaction between RAD52 and RPA-ssDNA complex (E.) by Z58-87. Complexes at the starting point of inhibitor titrations are depicted schematically on the top of each graph with respective FRET values indicated. The data shown as average±standard deviation for at least three independent measurements. Where invisible, the error bars are smaller than the respective symbols. Calculated IC₅₀ values are shown under each curve. F. Diagram of the two defective BRCA2 alleles of the EUFA423 cell line. In HA, the BRCA2 deficiency in complemented by expression of the full-length protein. G. Cell viability in the presence of various concentrations of Z58-87 was evaluated using CellTiter-Glo assay. The data are plotted as average±standard deviation for at least three independent measurements. Hundred percent live cells corresponds to DMSO only control.

### DETAILED DESCRIPTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein a wavy line “-~” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine, followed by the removal of 1 or more atoms from the second functional group to create the open valence.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; and (C₂-C₆)alkanoyloxy can be acetoxy, propanoyloxy, butanoyloxy, isobutanoyloxy, pentanoyloxy, or hexanoyloxy.

A specific value for X is (C₁)alkenyl that is optionally substituted with one or more fluoro.

A specific value for X is —CH₂—.

A specific value for Y is ═O.

A specific value for ring A is ring A that is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

A specific value for ring B is ring B that is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

A specific value for R¹ is (C₁-C₃)alkyl or (C₁-C₃)alkoxy, wherein any (C₁-C₃)alkyl, and (C₁-C₃)alkoxy is optionally substituted with one or more groups independently selected from the group consisting of halo.

A specific value for R¹ is (C₁-C₃)alkoxy that is optionally substituted with one or more groups independently selected from the group consisting of halo.

A specific value for R¹ is methoxy that is optionally substituted with one or more groups independently selected from the group consisting of halo.

A specific value for R¹ is methoxy.

A specific value for R² is (C₁-C₃)alkyl, or (C₁-C₃)alkoxy, wherein any (C₁-C₃)alkyl, and (C₁-C₃)alkoxy is optionally substituted with one or more groups independently selected from the group consisting of halo.

A specific value for R² is (C₁-C₃)alkyl that is optionally substituted with one or more groups independently selected from the group consisting of halo.

A specific value for R² is methyl that is optionally substituted with one or more groups independently selected from the group consisting of halo.

A specific value for R² is methyl.

A specific value for R³ is nitro.

A specific value for ring C is ring C that is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

A specific value for ring D is ring D that is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

In one embodiment, the compound of formula (II) or the salt thereof is not compound Z56:

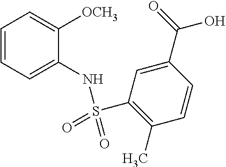 [SMILES: COc1ccccc1NS(=O)(=O)c1cc(C(=O)O)ccc1C]

or a salt thereof.

In one embodiment, the invention provides a pharmaceutical composition comprising the compound Z56:

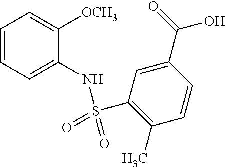 [SMILES: COc1ccccc1NS(=O)(=O)c1cc(C(=O)O)ccc1C]

or a pharmaceutically acceptable salt thereof and a pharmaceutically acceptable excipient.

A specific pharmaceutical composition comprises a compound of formula (I) or formula (II), or a pharmaceutically acceptable salt thereof, a pharmaceutically acceptable excipient; and a PARP inhibitor.

Processes for preparing compounds of formula I are provided as further embodiments of the invention and are illustrated by the following procedures in which the meanings of the generic radicals are as given above unless otherwise qualified.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with radiation and other therapeutic agents, for example, other agents that are useful for the treatment of cancer. Examples of such agents include PARP inhibitors, platinum containing chemotherapeutic compounds (cisplatin, carboplatin, oxaliplatin), DNA damaging agents such as doxorubicin, melphalan, bendamustine, and Gemcitibine.

In non-limiting examples, the compounds useful within the invention may be used in combination with one or more of the following therapeutic agents: Erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), pemetrexed (ALIMTA®, Eli Lilly), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, rapamycin, oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GaxoSmithKline), canfosfamide (TELCYTA®, Telik), thiotepa and cyclosphosphamide (CYTOXAN®, NEOSAR®).

Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat cancer. Compounds of the invention can also be administered to treat cancer in combination with other therapies, such as, for example, radiation therapy.

The invention will now be illustrated by the following non-limiting Examples.

### EXAMPLES

### Examples 1-5

The compounds of Examples 1-5 were prepared using the following general procedure.

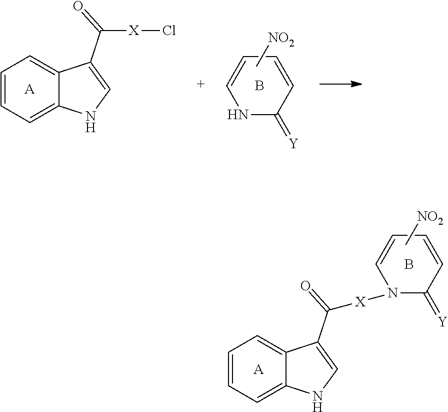

The requisite pyrid-2-one (1 equivalent) was dissolved DMF. Cesium carbonate (1.2 equivalents) was added. The reaction mixture was stirred at room temperature for 15 minutes and an appropriate 2-chloro-1-(1H-indolyl)ethan-1-one (1 equivalent) was added. The reaction mixture was stirred at room temperature for 16 hours and diluted with cold water. The resulting precipitate was filtered and washed twice with water. The material was purified by HPLC.

### Example 1. Preparation of Z99

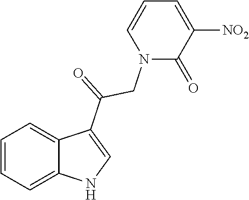 [SMILES: O=C(Cn1cccc([N+](=O)[O-])c1=O)c1c[nH]c2ccccc12]

Chemical Formula: C₁₅H₁₁N₃O₄

MW: 297.3 Exact Mass: 297.075 RT: 5.8 min.

[M−H]⁻ at m/z 296.0675 (100%), 297.0709 (16.3%), 298.0735 (1.4%)

[M+H]⁺ at m/z 298.0820 (100%), 299.0854 (15.7%), 300.0882 (1.4%)

### Example 2. Preparation of Z5862216254 (Z58-54)

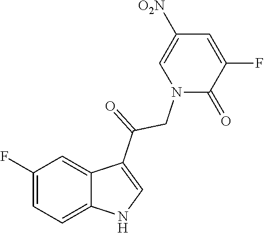 [SMILES: O=C(Cn1cc([N+](=O)[O-])cc(F)c1=O)c1c[nH]c2ccc(F)cc12]

Chemical Formula: C₁₅H₉F₂N₃O₄

MW: 333.2 Exact Mass: 333.2471 RT: 7.7 min.

[M−H]⁻ at m/z 332.0489 (100%), 333.0520 (16.0%), 334.0549 (1.6%)

[M+H]⁺ at m/z 334.0630 (100%), 335.0665 (15.2%), 336.0688 (1.4%)

### Example 3. Preparation of Z5862209772 (Z58-72)

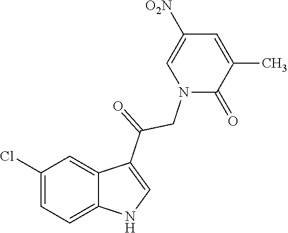 [SMILES: Cc1cc([N+](=O)[O-])cn(CC(=O)c2c[nH]c3ccc(Cl)cc23)c1=O]

Chemical Formula: C₁₆H₁₂ClN₃O₄

MW: 345.7 Exact Mass: 345.0516 RT: 9.4 min.

[M−H]⁻ at m/z 344.0449 (100%), 346.0417 (31.4%), 345.0482 (17.4%)

[M+H]⁺ at m/z 346.0590 (100%), 348.0559 (29.4%), 347.0625 (16.0%)

### Example 4. Preparation of Z5862135983 (Z58-83)

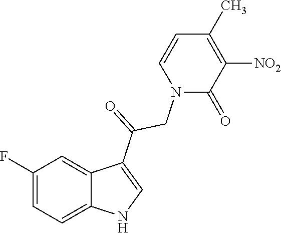 [SMILES: Cc1ccn(CC(=O)c2c[nH]c3ccc(F)cc23)c(=O)c1[N+](=O)[O-]]

Chemical Formula: C₁₆H₁₂FN₃O₄

MW: 329.3 Exact Mass: 329.0812 RT: 7.9 min.

[M−H]⁻ at m/z 328.0742 (100%), 329.0774 (17.1%), 330.0802 (1.8%)

[M+H]⁺ at m/z 330.0884 (100%), 331.0918 (16.8%), 332.0946 (1.6%)

### Example 5. Preparation of Z5862219887 (Z58-87)

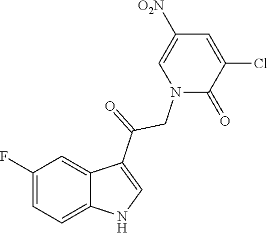 [SMILES: O=C(Cn1cc([N+](=O)[O-])cc(Cl)c1=O)c1c[nH]c2ccc(F)cc12]

### Examples 6-7

The compounds were prepared using the following general procedure.

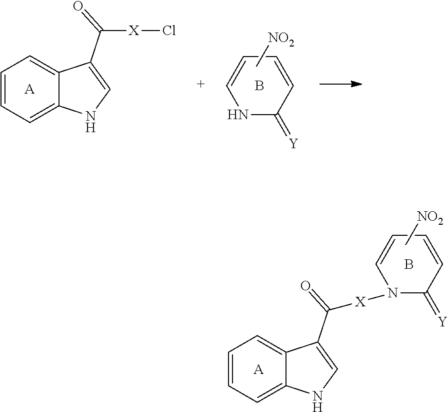

The requisite pyrid-2-one (1 equivalent) was dissolved DMF. Cesium carbonate (1.2 equivalents) was added. The reaction mixture was stirred at room temperature for 15 minutes and an appropriate 2-chloro-1-(1H-indolyl)ethan-1-one (1 equivalent) was added. The reaction mixture was stirred at room temperature for 16 hours and diluted with cold water. The resulting precipitate was filtered and washed twice with water. The material was purified by HPLC.

### Example 6. Preparation of Z5862148469 (Z58-69)

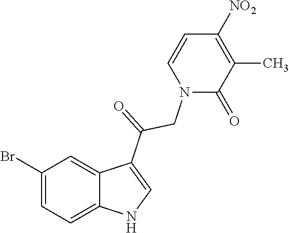 [SMILES: Cc1c([N+](=O)[O-])ccn(CC(=O)c2c[nH]c3ccc(Br)cc23)c1=O]

Chemical Formula: C₁₆H₁₂BrN₃O₄

MW: 390.2 Exact Mass: 389.0011 RT: 9.6 min.

[M−H]⁻ at m/z 387.9943 (100%), 389.9923 (98.9%), 388.9978 (17.3%)

### Example 7. Preparation of Z5862231762 (Z58-62)

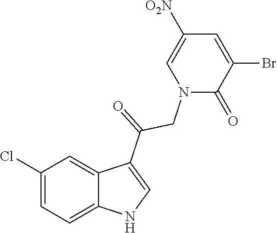 [SMILES: O=C(Cn1cc([N+](=O)[O-])cc(Br)c1=O)c1c[nH]c2ccc(Cl)cc12]

Chemical Formula: C₁₅H₉BrClN₃O₄

MW: 410.6 Exact Mass: 408.9465 RT: 9.8 min.

[M−H]⁻ at m/z 409.9370 (100%), 407.9394 (78.0%), 411.9341 (24.2%)

### Example 8. Preparation of Z56

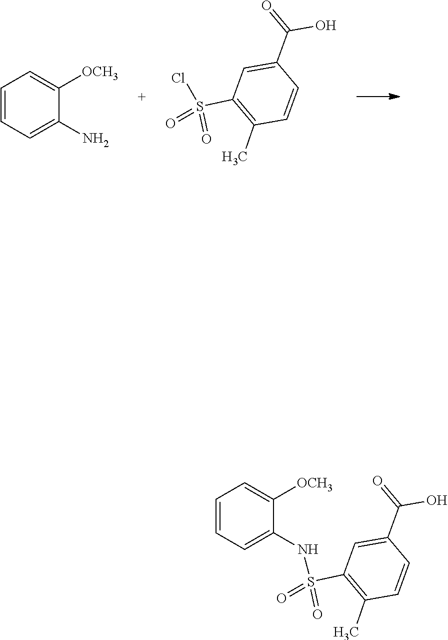 [SMILES: COc1ccccc1N ; Cc1ccc(C(=O)O)cc1S(=O)(=O)Cl ; COc1ccccc1NS(=O)(=O)c1cc(C(=O)O)ccc1C]

The compound can be prepared using a procedure similar to that described by Silvio Aprile, et al., ACS Med. Chem. Lett. 2021, 12, 640-646 (S13).

Chemical Formula: C₁₅H₁₄NO₅S

MW: 320.3 Exact Mass: 320.0593 RT: 7.3 min.

[M]⁻ at m/z 320.0597 (100%), 321.0625 (16.9%), 322.0554 (4.3%)

[M+2H]⁺ at m/z 322.0739 (100%), 323.0774 (16.2%), 324.0697 (4.3%)

### Example 9. Biological Testing

### Material and Methods

Proteins: The 6×His-tagged human RAD52 proteins (wild type and mutants) and untagged human RPA were expressed and purified as previously described in (Hengel S R, eLife. 2016; 5; Grimme J M, et al., Nucleic Acids Res. 2010; 38(9):2917-30; and Henricksen L A, et al., The Journal of biological chemistry. 1994; 269(15):11121-32). RPA and RAD52 protein concentrations were determined by measuring absorbance at 280 nm using extinction coefficients of 88,830 M⁻¹ cm⁻¹ and 40,470 M⁻¹ cm⁻¹, respectively.

Cell lines: EUFA423F cells that are derived from a Fanconi Anemia patient with complementation group D1 and have biallelic mutations (7691 insAT and 9900 insA) in BRCA2 that result in two different truncated forms of BRCA2 (Howlett N G, et al., Science (New York, NY). 2002; 297(5581):606-9) and its counterpart complemented with expression vector for full-length BRCA2 (EUFA HA) were a generous gift from Dr. Simon Powell, Molecular Biology Program, Radiation Oncology, Sloan-Kettering Cancer Center, New York, NY).

FRET-based DNA binding and inhibition assays: FRET-based that monitor the RAD52-ssDNA, RPA-ssDNA and RAD52-RPA-ssDNA binding were carried out as previously described (Hengel S R, eLife. 2016; 5; Grimme J M, et al., Nucleic Acids Res. 2010; 38(9):2917-30; and Grimme J M, Spies M. FRET-based assays to monitor DNA binding and annealing by Rad52 recombination mediator protein. Methods in molecular biology (Clifton, NJ). 2011; 745:463-83) using Cary Eclipse Spectrofluorimeter (Agilent) at 25° C., in 5 mm quartz cuvettes, and in buffer containing 30 mM Tris-Acetate (pH 7.5) and 1 mM DTT. Cy3 dye was excited at 530 nm and its emission was monitored at 565 nm. Emission of Cy5 acceptor fluorophore excited through the energy transfer from Cy3 donor is monitored at 660 nm simultaneously with emission of Cy3 dye. Both the excitation and the emission slits were set to 10 nm. All experiments were carried out using 1 nM (molecules) Cy3-dT30-Cy5 ssDNA. The optimal concentrations of the RAD52 (6 nM) and RPA (1 nM) proteins were selected by titrating the respective protein into 1 nM Cy3-dT30-Cy5 solution, and chosen based on the largest separation of the FRET values corresponding to the protein bound and free DNA.

The inhibition of the RAD52-ssDNA, RPA-ssDNA and RAD52-RPA-ssDNA complexes was evaluated by titrating each compound to the respective complex, measuring Cy3 and Cy5 fluorescence, and calculating FRET as previously described (Hengel, S. R., et al., eLife, 5).

Spectral properties of many compounds overlapped with the Cy3 and Cy5 fluorescence. To correct the calculated FRET values for the compound signal, the fluorogenic compounds were titrated into buffer and into solution of 1 nM Cy3-dT30-Cy5. For all tested compounds, the change in the signal in the Cy3 and Cy5 channels with and without Cy3/Cy5-labeled DNA present was the same. It was concluded that the compounds do not change the ssDNA conformation and are unlikely to interact with ssDNA. To account for the dye fluorescence, for each compound concentration, the apparent change in FRET of the ssDNA in the presence the compound was subtracted from the respective FRET signal of the RAD52-ssDNA, RPA-ssDNA or RAD52-ssDNA-RPA complex at the same inhibitor concentration. The IC₅₀ values were calculated by fitting the corrected FRET data to the four-parameter dose response model using GraphPad Prism. All data are shown as average for at least three independent experiments ±standard deviation. The IC₅₀ values are presented with their respective fitting error.

Cell proliferation assay: Selective toxicity of the compounds towards human BRCA2 deficient cells was tested in EUFA423F and its complement EUFA423F HA using CellTiter-Glo® Luminescent Cell Viability Assay (Promega, Madison, WI). The cell lines were maintained in DMEM (Gibco/ThermoFisher Scientific, Waltham, MA) supplemented with 10% FBS and 10 mM HEPES (Gibco/ThermoFisher Scientific, Waltham, MA). EUFA423F HA cells were routinely supplemented with G418 in order to maintain the BRCA2 expression plasmid. Both cell lines were seeded in sterile tissue culture treated, white opaque 384-well plates (Perkin Elmer Inc., Waltham, MA) 3000 cells/well for EUFA423F HA and 2500 cells/well for EUFA423F) and allowed to adhere for 24 hours. Compounds dissolved in DMSO were added in the concentration range of 0-100 μM. Controls were treated with equivalent DMSO volumes. The cells were treated with compounds for 72 hours (to ensure that all cells experienced at least two S-phases). Post treatment, the cells were washed, treated with the CellTiter-Glo® reagent was added and the plate was analyzed as using a Gen5 (ver2.1) luminosity plate reader. The luminescence that reports on ATP generation by the surviving cells was normalized for the DMSO treated control as 100%. The normalized luminescence (average±standard deviation for at least three independent measurements obtained with different cell passages) was plotted in GraphPad Prism as a function of compound concentration.
Clonogenic Survival: EUFA423F and EUFA423F HA cells were seeded in a 96 well plate. Compounds dissolved in DMSO were added to final concentrations of 0, 50, and 100 μM for the compound of Example 8 and the compound of Example 1, and 0, 5, and 10 μM for the compounds of Examples 2-7. The cells were treated with compounds for 72 hours. Post treatment, cell cultures were harvested by collecting all non-adherent cells in the well, trypsinization of all adherent cells, combining, pelleting and then resuspending in fresh media. Cultures treated with DMSO only were subsequently counted using a hemocytometer and plated at a density of 2000, 200 and 20 cells per well in 6-well culture plates, equal volumes of cultures were plated each inhibitor concentration. Each treatment and cell density conditions for both cell lines were plated in triplicate. After expanding the colonies for a period of 8 days, cultures were fixed with a solution of 70% ethanol and crystal violet for clonogenic survival analysis. Pictures of individual wells were taken using GelCount (Version 1.3.0.4) by Oxford Optronix (Abingdon UK). A colony had to contain at least 50 cells to be counted. The number of surviving colonies following treatment are presented as a percentage of number of colonies in the experiment where the cells were treated with DMSO only.

### Results

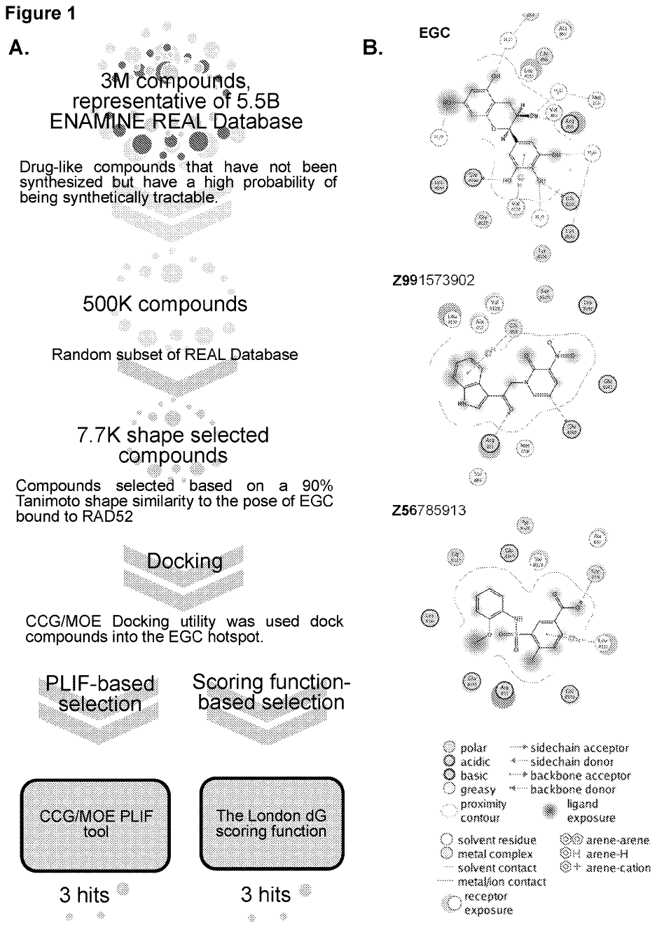

FIG. 1 shows the computational workflow (FIG. 1A) that led to selection of Z99 (Compound 1) and Z56 (Compound 8). FIG. 1B shows computational placement of these two compounds into the ssDNA binding grove of RAD52, and specifically to the EGC binding site.

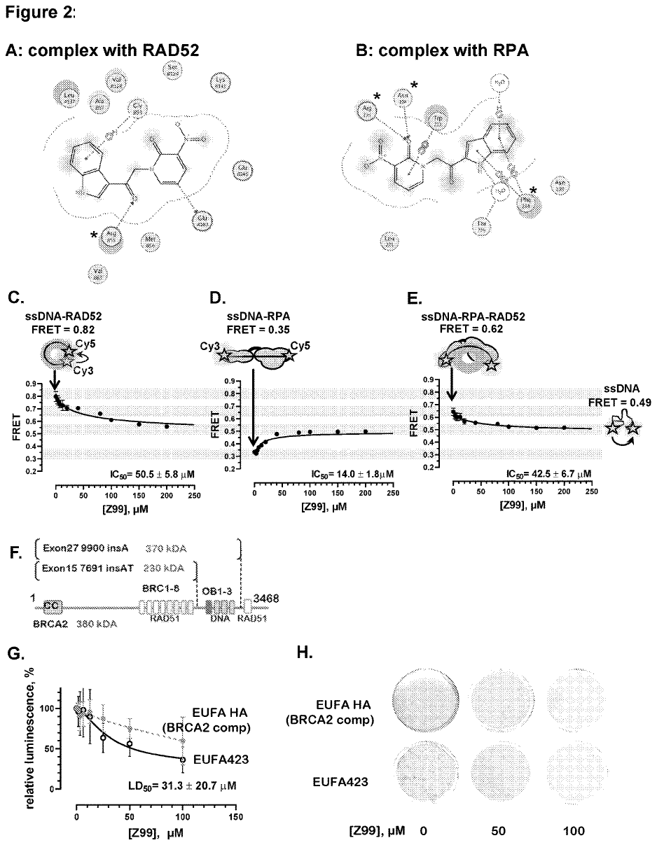

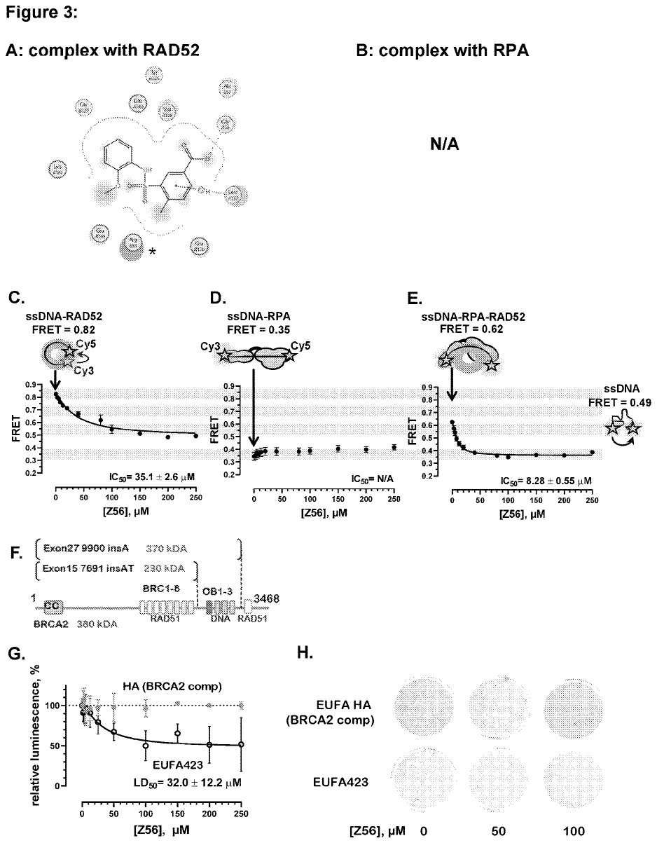

While both compounds were promising RAD52 inhibitors, Z56 was specific to RAD52, while Z99 was more promiscuous also inhibiting RPA. FIGS. 2 and 3 show the detailed characterization of these two novel compounds.

Increasing concentrations of both Z56 and Z99 applied to the RAD52 ssDNA complex result in the FRET decrease from the values (0.82) corresponding to the wrapped RAD52-ssDNA complex to the values corresponding to free ssDNA (0.49) (FIGS. 2C and 3C).

Addition of Z56 does not change the FRET values of the RPA-ssDNA complex (0.35) (FIG. 3D) and decreases the FRET values of the RPA-ssDNA-RAD52 complex (0.62) to the values corresponding to the RPA-ssDNA complex (FIG. 3E). In contrast, titration of Z99 into both RPA-ssDNA and RPA-ssDNA-RAD52 complex yields FRET values corresponding to the free ssDNA (FIGS. 2D and 2E). Because of their IC₅₀ values and difference in specificity, these two compounds were selected for the cell-based analyses discussed below.

Z56 and Z99 Inhibitors Bind within the Inner Binding Groove of the RAD52 Oligomeric Ring.

Biochemical and structural studies identified two distinct DNA binding sites in the RAD52 protein (Saotome M, et al., iScience. 2018; 3:50-62; Kagawa W, et al., The Journal of biological chemistry. 2008; 283(35):24264-73; and Lloyd J A, et al., J Mol Biol. 2005; 345(2):239-49). The “inner” or “primary” binding site is a narrow groove that spans the circumference of the protein and accommodates ssDNA. A specific pocket within this groove was used in our computational workflow. Positively charged residues deep in the groove make electrostatic contacts with the DNA backbone. Notably, of the key ssDNA contacts, R55 makes strong interactions with both Z56 and Z99. The “outer” or “secondary” DNA binding site of RAD52 binds both ssDNA and dsDNA and may provide a platform for DNA annealing (Saotome M, et al., iScience. 2018; 3:50-62; and Kagawa W, et al., The Journal of biological chemistry. 2008; 283(35):24264-73)

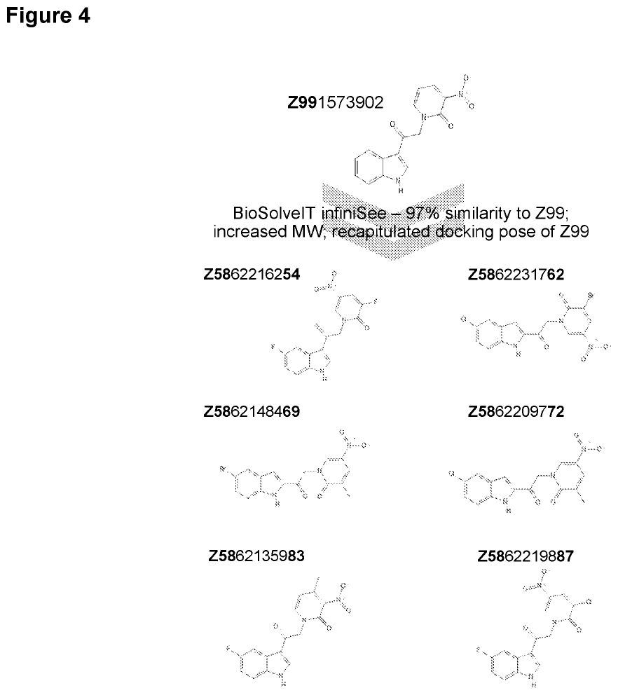

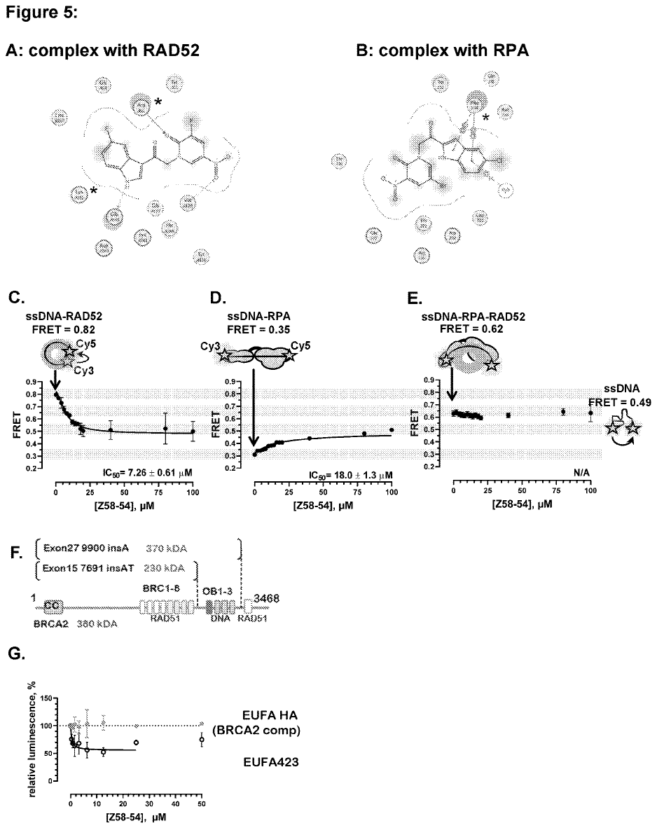

Two mutants were purified, inner binding defective (IB, K152A/R153A, FIG. 5A and FIG. 5B) mutant, and outer binding defective mutant (OB, K102A/K103A/K169A/R173A, FIG. 5). Both mutants retained the capacity to bind ssDNA and RPA-ssDNA complex but distorted the DNA to a lesser degree than the wild type RAD52 (FIG. 5C and FIG. 5D). Both Z56 and Z99 were able to inhibit ssDNA binding to IB and OB mutants. Notably, the IC₅₀ value for Z56 inhibition of the wild type and IB mutant RAD52 were within the fitting error; in contrast, IC₅₀ value for OB mutant, which can only engage the inner binding site for DNA binding was 6-fold lower. This correlates well with the computed binding position of this compound (FIG. 4E). Notably, the IC₅₀ values for Z56 inhibiting the RPA-ssDNA-RAD52(mutant) complex for both mutants were within fitting error of the corresponding values for RAD52(mutant)-ssDNA complex, while for the wild type protein the IC₅₀ value for the RPA-ssDNA-RAD52 complex was 6-fold lower than for the RAD52-ssDNA complex, suggesting plasticity of the ssDNA binding within the RAD52 oligomeric ring (Table 1). Z99 had enhanced efficiency for both IB and OB mutant compared to the wild type RAD52, but similarly to Z56 had 6-fold increased efficiency against OB mutant compared to IB mutant.

### Z56 and Z99 Selectively Kill BRCA2-Mutant Cells but Promiscuity of Z99 Causes Toxicity

Z56 and Z99 had sufficiently low IC₅₀ values to be tested in cell-based experiments. Notably, these compounds represent both computational approaches, PLIFs and docking, and also represent compounds that are specific to RAD52 (e.g. Z56) or can promiscuously inhibit RPA (e.g. Z99). Cell-based viability experiments were carried out using CellTiter-Glo® Luminescent Cell Viability Assay (see Materials and Methods for details) and an isogenic pair of cell lines: EUFA423F is a Fanconi Anemia cell line that harbors biallelic mutations (7691 insAT and 9900 insA) in BRCA2 gene resulting in two different truncated forms of BRCA2 (Howlett N G, et al., Science (New York, NY). 2002; 297(5581):606-9) and its counterpart complemented with expression vector for full-length BRCA2 (FIG. 2F). The two BRCA2 truncations in EUFA423F are missing the C-terminal RAD51 binding region important for the BRCA2 function at stalled replication forks (Esashi F, et al., Nature. 2005; 434(7033):598-604; Feng W, Jasin M. Nature communications. 2017; 8(1):525; Lomonosov M, et al., Genes Dev. 2003; 17(24):3017-22; and Schlacher K, et al., Cell. 2011; 145(4):529-42), and the GB-fold containing region important in BRCA2-ssDNA interaction (Paul M W, et al., eLife. 2021; 10; Esashi F, G et al., Nature structural & molecular biology. 2007; 14(6):468-74; Yang H, et al., Science (New York, NY). 2002; 297(5588):1837-48; and Zhao W, et al., Mol Cell. 2015; 59(2):176-87) (FIG. 1C). EUFA423F are sensitive to PARP inhibitor velaparib (ABT-888) and are believed to represent a good BRCA-deficient cell model (Clark C C, et al., Mol Cancer Ther. 2012; 11(9):1948-58). The complemented cell line (HA) has a fully functional BRCA2, and is resistant to veliparib (Clark C C, et al., Mol Cancer Ther. 2012; 11(9):1948-58) and DNA cross-linking agent mitomycin C (Howlett N G, et al., Science (New York, NY). 2002; 297(5581):606-9).

Z56 (FIG. 3G) and Z99 (FIG. 2G) were capable of selectively targeting BRCA2 deficient cells. While both Z56 and Z99 were selectively toxic to BTCA2-deficient EUFA423F cells, high concentrations of Z99 affected the viability of BRCA2-complementd HA cells. The reason for this toxicity is likely the capacity of Z99 to inhibit RPA-ssDNA interaction. In contrast, Z56, which had no effect on the RPA-ssDNA complex in FRET-based assays was also selective in cell viability experiments, leaving the growth of BRCA2-complemented HA cells unaffected, even at high (250 μM) concentrations of the compound. The IC₅₀ value for the EUFA423F viability experiments using Z56 (32.0±12.1 μM) were very similar to the IC₅₀ value for the disruption of the RAD52-ssDNA complex in FRET-based studies. The effect on the Z56 and Z99 compounds on cell viability was also confirmed in clonogenic survival assays. FIGS. 2H and 3H show representative wells ceded by the cells pre-treated for 72 hours with the respective concertation of inhibitor or an equivalent amount of DMSO. Note that both EUFA423F and EUFA423F HA cells form diffuse colonies with variable number of cells and large distances between the individual cells.

### Expansion of the Z99 Scaffolds Improves Activity Against RAD52, but not RPA, and Efficacy in Selective Killing of BRCA2-Mutant Cells:

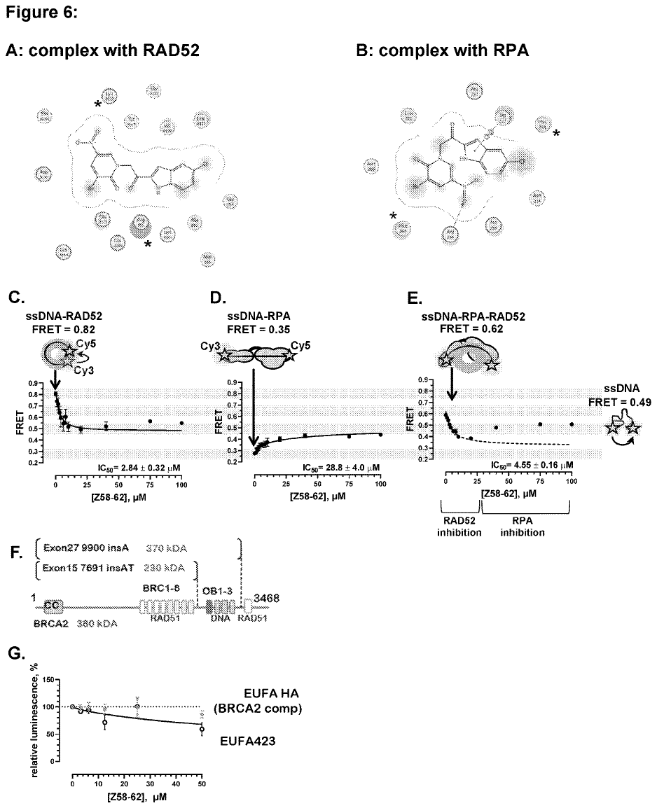

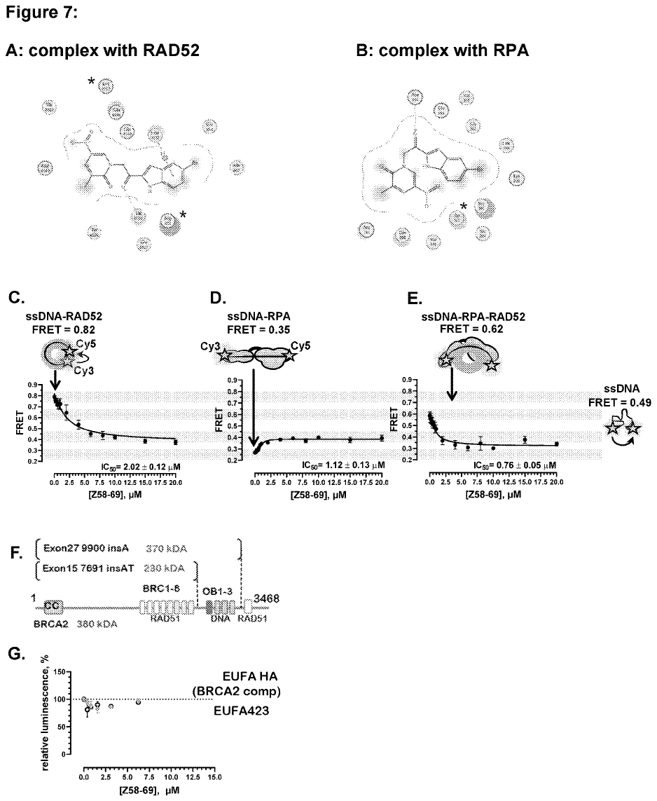

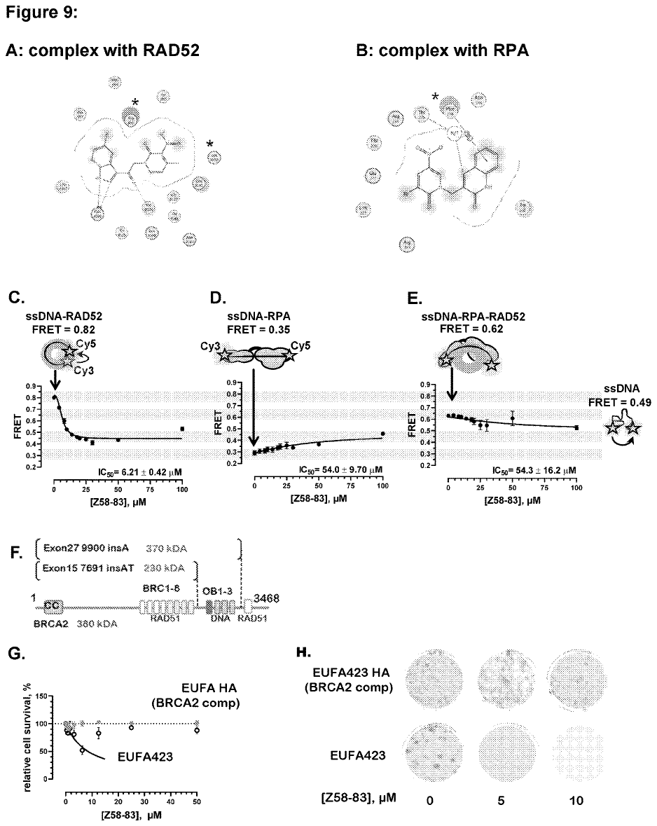

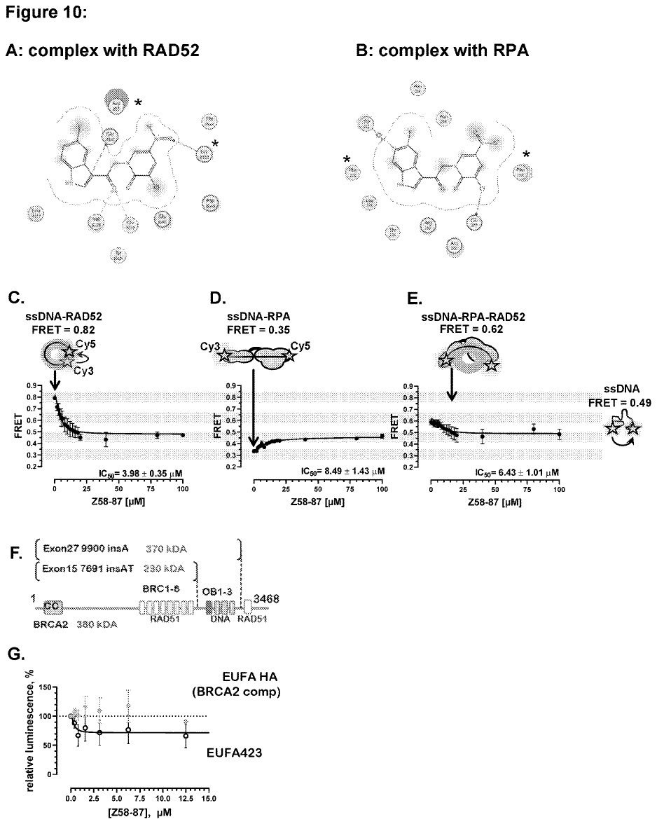

Six new compounds were generated based on the Z99 scaffold. InfiniSee (BiosolveIT) was used to identify compounds with diverse scaffolds and a 97% shape overlap with Z99 from the Enamine REAL space (FIG. 4). FIGS. 5-10 show characterization of these compounds using the same methodologies as described for Z99 and Z56. These additional compounds showed a marked improvement in their ability to inhibit the RAD52-ssDNA interactions. The IC₅₀ values for these compounds ranged between 2 and 8 μM, 6-40-fold improvement compared to Z99 (FIGS. 5C-10C). While all of the compounds retained their ability to inhibit the RPA-ssDNA binding, three compounds had higher IC₅₀ than Z99, two compounds were similar in potency to Z99 for RPA inhibition, while only one had lower IC₅₀ for RPA (Table 1). Thus, these compounds can provide a broader therapeutic window in cell based experiments.

Notably, one of the compounds, Z5862216254, while effective in disrupting the RAD52-ssDNA interaction (IC₅₀ of 7.26±0.61 μM), and slightly more poor disruption of the RPA-ssDNA complex (IC₅₀ of 18.0±1.3 μM), was unable to dislodge RAD52 from the RPA-ssDNA-RAD52 complex. It was nevertheless effective and specific in cell viability assays (FIG. 3D). All compounds, except Z5862148469 were effective and specific in cell viability assays at low compound concentrations. If fact, most of the compound were more effective than PARP inhibitor velaparib (41). At higher compound concentration, these compounds aggregated in the media losing efficacy. Clonogenic survival experiments confirmed the efficacy and improved specificity of the expanded compounds (FIGS. 5G-10G).

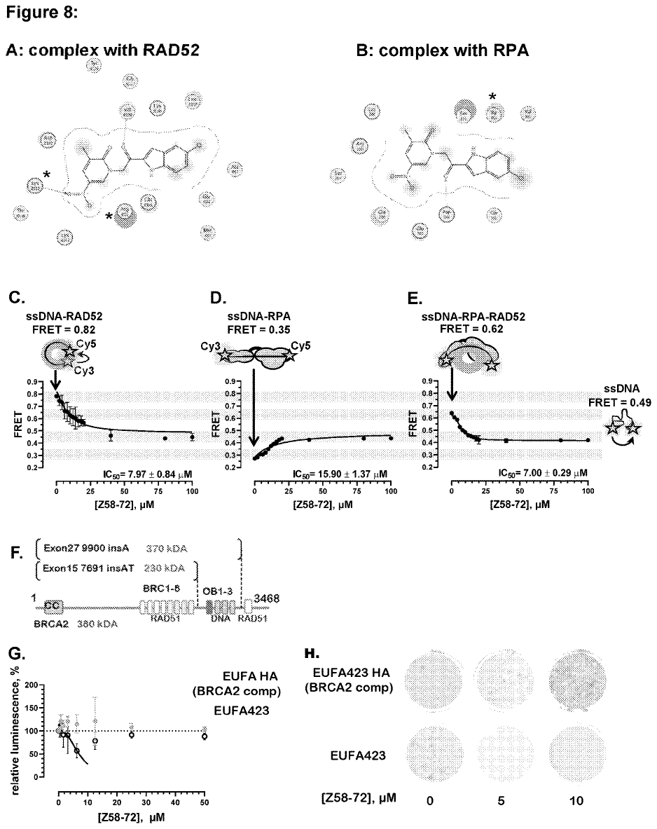

To understand the nature of the RPA-ssDNA complex disruption, molecular docking alongside EGC and EGCG, a known RPA binder, was evaluated. Human RPA interacts with ssDNA through four OB folds, known as DNA binding domains (DBD) A, B, C and D. The four DBDs and their modes of ssDNA binding are structurally similar (18). Therefore, PDB was used: 1JMC structure of the DBD-A and DBD-B of human RPA in complex with ssDNA (44) to evaluate the interaction between RPA-displacing compounds and RPA (FIG. 8A). The most favourable binding pose of Z99 vis-à-vis EGCG is shown in FIG. 8B and FIG. 8C. In this pose, stacking occurs between the Z99 and the aromatic residues W212 and F238 of the RPA DBD-A. Note that F238 is one of the key aromatic residues important for RPA function (Hass C S, et al., The Journal of biological chemistry. 2012; 287(6):3908-18). There is also hydrogen bonding donation from R216 and N214 (ssDNA interacting residues) to the carbonyl oxygen of Z99. The same contacts were also present in the EGCG-RPA complex. In addition, the EGCG pocket also contains R210 and R234. Interestingly, among the compounds Z99 itself was the strongest RPA binder from the docking simulations, and its heavier derivatives did not replicate the same excellent stacking and hydrogen bonding indicated above (Compare FIGS. 2B, 5B-10B). Z5862216254, Z5862231762, and Z5862219887, while although binding in the same area as Z99, completely lacked stacking with F238 and protruded out of the sub-pocket, also forming poorer stacking with W212. Notably, the binding pocket accommodating Z5862219887 also included F269, the second aromatic residue of the ssDNA binding cleft of DBD-A. Z5862148468 and Z5862209772 could not even bind in the Z99 subpocket, binding instead at the DBD-A/DBD-B interface close to the W361, the first aromatic residue of the DBD-B. Z58-83 was able to bind in the same subpocket as Z99, with stacking to W212, but weaker interaction with F238.

Thus, it is fortunate that the steric requirements for Z99 derivatives are challenging, and that their larger volumes are not allowing the optimal stacking and hydrogen bonding seen with Z99. This nicely parallels what is seen in the experimental in vitro studies (discussed above), in which, although RAD52 potency is increased with the Z99 derivatives, one does not see this mirrored in terms of disruption of the RPA-ssDNA complex.

### DISCUSSION

The invention provides drug-like small molecule inhibitors of an attractive anticancer drug target, human DNA repair protein RAD52. Certain compounds are specific to RAD52 over other ssDNA binding proteins, such as RPA, whose inhibition may be generally toxic to cells. Z56 and Z99 were especially attractive as they showed activity in cell viability assays (FIG. 1). Z56 was specific to RAD52 over RPA in vitro and showed toxicity only towards BRCA2 mutant cells. In contrast, Z99 was more promiscuous, inhibited RPA in vitro and had some toxicity towards BRCA2-complemented cells.

Docking of Z99 and Z56 to DBD-A/B of the RPA70 showed that Z99 strongly binds to a deep pocket on the RPA DBD-A, which is also the location of the top docking pose for EGCG (FIG. 8). The Compounds of Examples 2-7 were found to inhibit RAD52 with IC₅₀ values in low μM range, with several of the compounds being as active in cell-based assays as a PARP inhibitor veliparib, and excellent discrimination between BRCA2-mutant and complemented cells.

TABLE 1
Data For Representative Compounds

| | | | | | toxicity |
|---|---|---|---|---|---|
| | | | inhibition of the | | towards |
| | inhibition of the | inhibition of the | RPA-SSDNA- | toxicity | EUFA423 |
| | RAD52-SSDNA | RPA-SSDNA | RAD52 | towards | HA (BRCA2- |
| | interaction, | interaction, | complex, | EUFA423 | complemented |
| Example | IC₅₀ (μM) | IC₅₀ (μM) | IC₅₀ (μM) | cells | cells) |
| 1 | 50.5 ± 5.8 | 14.0 ± 1.8 | 42.5 ± 6.7 | yes | yes |
| 2 | 7.26 ± 0.61 | 18.0 ± 1.3 | N/A | yes | no |
| 7 | 2.84 ± 0.32 | 28.87 ± 4.0 | 4.55 ± 0.16 | yes | no |
| 6 | 2.02 ± 0.12 | 1.12 ± 0.13 | 0.76 ± 0.05 | no | no |
| 3 | 7.97 ± 0.84 | 15.9 ± 1.4 | 7.00 ± 0.29 | yes | no |
| 4 | 6.21 ± 0.42 | 54.0 ± 9.7 | 54.3 ± 16.2 | yes | no |
| 5 | 3.98 ± 0.35 | 8.49 ± 1.43 | 6.43 ± 1.01 | yes | no |
| 8 | 35.1 ± 2.6 | N/A | 8.28 ± 0.55 | yes | no |

N/A—no activity

### Example 10. The Following Illustrate Representative Pharmaceutical Dosage Forms, Containing a Compound of Formula I (‘Compound X’), for Therapeutic or Prophylactic Use in Humans

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula (I) or (II): 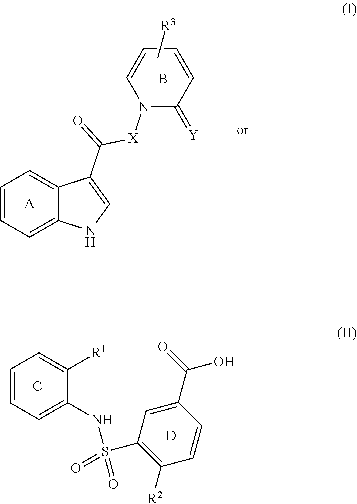
or a salt thereof, wherein:
X is (C₁-C₃)alkenyl that is optionally substituted with one or more fluoro;
Y is ═S or ═O;
R¹ is H, (C₁-C₆)alkyl, or (C₁-C₆)alkoxy, wherein any (C₁-C₆)alkyl, and (C₁-C₆)alkoxy is optionally substituted with one or more groups independently selected from the group consisting of halo;
R² is H, (C₁-C₆)alkyl, or (C₁-C₆)alkoxy, wherein any (C₁-C₆)alkyl, and (C₁-C₆)alkoxy is optionally substituted with one or more groups independently selected from the group consisting of halo;
R³ is nitro, halo, hydroxy, cyano, carboxy, (C₁-C₆)alkyl, (C₁-C₆)alkoxy, (C₁-C₆)alkoxycarbonyl, —NR^{a}R^{b}, —S(═O)₂R^{c}, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups;
ring A and ring B in formula (I) are each optionally substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy;
ring C and ring D in formula (II) are each optionally substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy;
R^{a} is H, (C₁-C₆)alkyl, (C₁-C₆)alkoxy, (C₁-C₆)alkoxycarbonyl, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups;
R^{b} is H, (C₁-C₆)alkyl, (C₁-C₆)alkoxy, (C₁-C₆)alkoxycarbonyl, or (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more fluoro groups; and
R^{c} is (C₁-C₆)alkyl that is optionally substituted with one or more fluoro groups.

2. The compound or salt of claim 1, which is a compound of formula (I) or a salt thereof.

3. The compound or salt of claim 1, wherein X is (Ci)alkenyl that is optionally substituted with one or more fluoro.

4. The compound or salt of claim 2, wherein X is —CH₂—.

5. The compound or salt of claim 2, wherein Y is ═O.

6. The compound or salt of claim 2, wherein ring A is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

7. The compound or salt of claim 2, wherein ring B in formula (I) is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

8. The compound or salt of claim 2, wherein R³ is nitro.

9. The compound or salt of claim 1, which is a compound of formula (II) or a salt thereof.

10. The compound or salt of claim 9, wherein R¹ is (C₁-C₃)alkyl or (C₁-C₃)alkoxy, wherein any (C₁-C₃)alkyl, and (C₁-C₃)alkoxy is optionally substituted with one or more groups independently selected from the group consisting of halo.

11. The compound or salt of claim 9, wherein R¹ is (C₁-C₃)alkoxy that is optionally substituted with one or more groups independently selected from the group consisting of halo.

12. The compound or salt of claim 9, wherein R¹ is methoxy that is optionally substituted with one or more groups independently selected from the group consisting of halo.

13. The compound or salt of claim 9, wherein R¹ is methoxy.

14. The compound of claim 9, wherein R² is (C₁-C₃)alkyl, or (C₁-C₃)alkoxy, wherein any (C₁-C₃)alkyl, and (C₁-C₃)alkoxy is optionally substituted with one or more groups independently selected from the group consisting of halo.

15. The compound of claim 9, wherein R² is (C₁-C₃)alkyl that is optionally substituted with one or more groups independently selected from the group consisting of halo.

16. The compound of claim 9, wherein R² is methyl that is optionally substituted with one or more groups independently selected from the group consisting of halo.

17. The compound of claim 9, wherein R² is methyl.

18. The compound of claim 9, wherein ring C is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

19. The compound of claim 9, wherein ring D is substituted with one or more groups independently selected from the group consisting of halo, hydroxy, cyano, carboxy, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy, wherein any, (C₁-C₆)alkyl, (C₂-C₆)alkenyl, (C₂-C₆)alkynyl, (C₃-C₆)cycloalkyl, (C₁-C₆)alkoxy, (C₁-C₆)alkoxycarbonyl, and (C₁-C₆)alkanoyloxy is optionally substituted with one or more groups independently selected from the group consisting of oxo (═O), halo, hydroxy, cyano, (C₃-C₆)cycloalkyl, and (C₁-C₆)alkoxy.

20. A pharmaceutical composition comprising a compound as described in claim 1 or a pharmaceutically acceptable salt thereof and a pharmaceutically acceptable excipient.

21-23. (canceled)

24. A method for treating cancer in an animal comprising administering a compound of formula (I) or formula (II) as described in claim 1 or a pharmaceutically acceptable salt thereof to the animal.

25-38. (canceled)
